(19) United States
(12) Patent Application Publication
(10) Pub. No.: US 2012/0100109 A1
(43) Pub. Date: Apr. 26, 2012

(54) Method for increasing the replication of oncolytic HSVs in highly resistant tumor cells using mTOR pathway and PI3K inhibitors

(76) Inventors: Xiaoliu Zhang, Houston, TX (US); Xinping Fu, Houston, TX (US)

(21) Appl. No.: 13/279,471

(22) Filed: Oct. 24, 2011

Related U.S. Application Data
(60) Provisional application No. 61/406,951, filed on Oct. 26, 2010.

(51) Int. Cl. A61K 35/76; C12N 5/09; A61P 35/00; C12N 7/00

(52) U.S. Cl. 424/93.6; 435/235.1; 435/325

(57) ABSTRACT

The present invention is directed to the administration of an HSV derived oncolytic virus and a PI3K/AKT/mTOR pathway inhibitor to treat various types of resistant tumors. Therapy-resistant tumor formation is one of the main causes for treatment failure in the clinic. The treatment methods and compositions disclosed herein sensitize resistant tumors to the treatment of herpes simplex virus (HSV)-based oncolytic virotherapy. Pre or co-treatment of resistant tumor cells with the mTOR inhibitor, rapamycin, or certain PI3K inhibitors, such as LY294002, can efficiently sensitize the tumors to HSV derived oncolytic viruses, whereby the replication and spread of the viruses are dramatically enhanced.

### CROSS-REFERENCES TO RELATED APPLICATIONS

This application claims priority to provisional application No. 61/406,951 filed on Oct. 26, 2010, which is herein incorporated by reference in its entirety.

### STATEMENTS AS TO RIGHTS TO INVENTIONS MADE UNDER FEDERALLY SPONSORED RESEARCH OR DEVELOPMENT

The U.S. Government has a paid-up license in this invention and the rights in limited circumstances to require the patent owners to license others on reasonable terms as provided for by the terms of grant Nos. 7R01CA132792-03 and 7R01CA106671-07 awarded by the National Institute of Health.

### FIELD OF THE INVENTION

The present invention is directed to methods and compositions to significantly increase the yield and dissemination of oncolytic Herpes simplex viruses (HSVs) in semipermissive or resistant tumor cells. The present invention also relates to the combined administration of PI3K/AKT/mTOR pathway inhibitors (e.g., rapamycin and LY294002) and a HSV-derived oncolytic virus (a virus that can selectively kill tumor cells) to either block or reverse the growth of tumors that are otherwise resistant to the therapeutic effect of either agent alone. This invention has important applications in potentiating the activity of oncolytic HSVs against difficult-to-treat human tumors and/or in preventing the emergence of resistant tumor cells during virotherapy.

### BACKGROUND OF THE INVENTION

Virotherapy has shown substantial promise as a new treatment modality for a broad range of human tumors (Russell, et al., Viruses as anticancer drugs, Trends Pharmacol. Sci. 2007; 28:326-33; Liu, et al., Clinical trial results with oncolytic virotherapy: a century of promise, a decade of progress, Nat. Clin. Pract. Oncol. 2007; 4:101-17). Oncolytic herpes simplex virus (HSV) is currently in phase III clinical trials for development as a novel therapeutic agent against a broad range of human tumors. Although results have been promising, clinical outcome is likely to be compromised by intrinsic and acquired resistance to HSV replication, leading us to test agents that may overcome this obstacle. The antitumor activity of an oncolytic virus derives mainly from its ability to replicate after it infects a tumor cell, with subsequent spread of the progeny virus to the nearby tumor cells. The resultant extent of tumor destruction often exceeds that achieved with many other types of cancer biotherapeutic agents (Thorne, et al., Oncolytic virotherapy: approaches to tumor targeting and enhancing antitumor effects, Semin. Oncol. 2005; 32:537-48). Consequently, the ability of an oncolytic virus to replicate robustly in tumor cells is a key factor in securing a favorable outcome from virotherapy (Everts, et al., Replication-selective oncolytic viruses in the treatment of cancer, Cancer Gene Ther. 2005; 12:141-61).

Herpes simplex virus (HSV) has a broad cell tropism, and oncolytic viruses derived from parental HSV strains can lyse tumor cells of many different tissue origins (Rabkin, et al, Replication-Competent Viruses for Cancer Therapy, Basel: Karger, 2001:1-45). Nonetheless, tumor cells that are resistant to HSV oncolysis are encountered from time to time and pose significant barriers to therapeutic outcomes. Several strategies have been proposed to overcome the resistance of tumor cells to HSV. It has been reported, for example, that serial passage of an oncolytic HSV (a γ34.5-deleted mutant) in resistant glioma cells can select for viral progeny that replicate more efficiently in the tumor cells and then show an enhanced antitumor effect against these resistant gliomas in vivo (Shah, et al., Serial passage through human glioma xenografts selects for a Deltagamma134.5 herpes simplex virus type 1 mutant that exhibits decreased neurotoxicity and prolongs survival of mice with experimental brain tumors, J. Virol. 2006; 80:7308-15). Prior art shows that although cyclophosphamide did not improve oncolytic HSV replication in the resistant Lewis lung carcinoma cells, its in vivo administration still enhanced the antitumor effect of the virotherapy (Li, et al., Coadministration of a herpes simplex virus-2 based oncolytic virus and cyclophosphamide produces a synergistic antitumor effect and enhances tumor-specific immune responses, Cancer Res. 2007; 67:7850-5).

Two groups have recently reported that rapamycin, an inhibitor of the mTOR (mammalian target of rapamycin) pathway, can increase the permissiveness of some resistant tumor cells to oncolytic myxoma virus or vesicular stomatitis virus (VSV). Stanford et al, for example, used rapamycin to pretreat human tumor cell lines that normally restrict myxoma virus replication and observed a striking increase in viral tropism and spread (Stanford, et al., Oncolytic virotherapy synergism with signaling inhibitors: Rapamycin increases myxoma virus tropism for human tumor cells, J. Virol. 2007; 81:1251-60). The enhanced replication of the myxoma virus in cells with a silenced mTOR pathway appeared to be linked to an increase in Akt kinase, suggesting that rapamycin could be used to improve the efficacy of oncolytic poxviruses in cancer treatment. Alain et al reported that rapamycin could significantly increase the replicative capability of an interferon (IFN)-sensitive VSV mutant (DeltaM51) in malignant glioma cells (Alain, et al., Vesicular stomatitis virus oncolysis is potentiated by impairing mTORC1-dependent type I IFN production, Proc. Natl. Acad. Sci. USA 2010; 107:1576-81). This enhancing effect apparently derived from the reduced inhibitory effect of type I IFNs on VSV (DeltaM51) replication once mTORC1 signaling is interrupted by rapamycin. More recently, rapamycin has been reported to enhance the therapeutic effect of a recombinant adenovirus carrying the gene encoding eukaryotic initiation factor 4E binding protein-1 (Mishra, et al., Adenovirus-mediated eukaryotic initiation factor 4E binding protein-1 in combination with rapamycin inhibits tumor growth of pancreatic ductal adenocarcinoma in vivo, Int. J. Oncol. 2009; 34:1231-40) and a newly developed mTOR inhibitor, Torin1, has been shown to enhance human cytomegalovirus replication (Moorman, et al., Rapamycin-resistant mTORC1 kinase activity is required for herpesvirus replication, J. Virol. 2010; 84:5260-9). However, none of these researchers have specifically investigated the effect of rapamycin on resistant tumor cells, nor did they look at oncolytic HSVs.

Previous studies failed to find any effective enhancement effect of rapamycin on replication of oncolytic HSVs in permissive tumor cells. For example, Some tumor cell lines are known to be fully permissive to the replication of oncolytic HSVs (Fu, et al., Expression of a fusogenic membrane glycoprotein by an oncolytic herpes simplex virus provides potent synergistic anti-tumor effect, Mol. Ther. 2003; 7:748-54; Fu, et al., A Mutant Type 2 Herpes Simplex Virus Deleted for the Protein Kinase Domain of the ICP10 Gene Is a Potent Oncolytic Virus, Mol. Ther. 2006; 13:882-90; Fu, et al., Potent systemic antitumor activity from an oncolytic herpes simplex virus of syncytial phenotype, Cancer Res. 2002; 62:2306-12). Baco-1 replicates to a high titer in these permissive tumor cells, reaching more than 1×10⁷ plaque-forming-units (pfu) per milliliter as illustrated in FIG. 1A. The presence of rapamycin in the culture medium does not significantly enhance the virus yield in any of the permissive tumor cell lines as shown in FIG. 1B, indicating that it lacks the capability to enhance the oncolytic effect of Baco-1 against tumor cells fully permissive to virus replication. As a result of these previous studies, it is likely that researchers concluded that rapamycin or other PI3K/Akt/mTOR pathway inhibitors such as LY294002 did not improve the effectiveness of HSV-derived virotherapy on any tumor cells. However, the present invention demonstrates that some of the PI3K/AKT/mTOR pathway inhibitors in fact have a significant impact on the efficacy of HSV-derived oncolytic treatment of resistant tumor cells.

Oncolytic viruses have the potential to improve clinical outcome for a spectrum of human tumors, but this promise is compromised by the existence or emergence of treatment-resistant tumor cells. Tumor cells become resistant to virotherapy for several reasons. They may lack receptors for a particular oncolytic virus, as reported for adenovirus-based oncolytic viruses (Van Beusechem, et al., Conditionally replicative adenovirus expressing a targeting adapter molecule exhibits enhanced oncolytic potency on CAR-deficient tumors, Gene Ther. 2003; 10:1982-91), but this mechanism is unlikely to apply to oncolytic HSVs, which rely on more ubiquitous cellular receptors for entry (Spear, Herpes simplex virus: receptors and ligands for cell entry, Cell Microbiol. 2004; 6:401-10). Instead, tumor cell resistance to HSV-based oncolytic viruses probably reflects failure of virus replication once the cell has been infected. The present invention overcomes this known limitation by using drugs such as rapamycin or LY294002 to release the restriction placed on oncolytic HSV replication in resistant tumor cells, therefore increasing the virus yield as well as the spread to nearby cells. The current invention clearly demonstrates that the combination of one or more than one drugs and an oncolytic HSV can potentiate viral replication in highly resistant tumor cells, leading to a significantly enhanced antitumor effect.

### SUMMARY OF THE INVENTION

The present invention addresses one of the core issues in the cancer treatment field. Therapy-resistant tumor formation is one of the main causes for reducing treatment effectiveness in the clinic. Methods and/or strategies to sensitize resistant tumors to a particular therapeutic modality can be extremely beneficial to the prognosis of cancer patients. The present invention discloses a method to sensitize resistant tumors to the treatment of herpes simplex virus (HSV)-based oncolytic virotherapy. This invention also emphasizes that HSV-derived oncolytic treatment of these semipermissive or resistant tumor cells with PI3K/AKT/mTOR pathway inhibitors, including but not limited to rapamycin or LY294002, can efficiently sensitize the cells to HSV-derived oncolytic viruses, i.e. increase tumor cell permissiveness to HSV-derived viruses. Without the drug treatment, HSV derived oncolytic viruses replicate and spread poorly in these tumor cells. When PI3K/AKT/mTOR pathway inhibitors are administered in an HSV-derived virotherapy, the replication and spread of the viruses are dramatically enhanced. For example, in animal models, administration of an inhibitor in an HSV-based virotherapy can efficiently shrink or even eradicate these tumors, while these components show little therapeutic effect if they are used individually. This discovery has a clear clinical value in forming a combinatorial treatment regimen for the treatment of resistant tumors.

### BRIEF DESCRIPTION OF THE DRAWINGS

FIG. 1. Rapamycin does not enhance oncolytic HSV replication in permissive tumor cells. Despite the dramatic enhancement effect of rapamycin on oncolytic HSV replication, it showed no effect on virus replication in three permissive tumor cells, as measured by either the actual virus yield (A) or the fold of virus yield change (B). Values represent the mean±SD of triplicate experiments.

FIG. 2. Rapamycin enhances oncolytic HSV replication in tumor cells that do not fully support the virus growth. FIG. 2A shows that the presence of rapamycin in the medium can increase the yield of oncolytic HSV by almost 6-fold in the resistant EC9706 tumor cell line. FIG. 2B shows the significant increase in the yield of oncolytic HSV in another two resistant tumor cells, MCF-7 and HeLa cells, in the presence of rapamycin in the culture medium. FIG. 2C shows the actual increase in the virus titer after rapamycin treatment to these three tumor cells. Virus yield, as determined by plaque assay. Values represent the mean±SD of triplicate experiments.

FIG. 3. Rapamycin and PI3K inhibitor LY294002 promote the spread of oncolytic HSV in semipermissive tumor cells and tumor cells that do not fully support the virus growth. In addition to the ability to increase the yield of oncolytic HSV in resistant tumor cells, both rapamycin (B) and LY294002 (C) can increase the spread of oncolytic HSV (Baco-1) among tumor cells in a monolayer (Baco-1 contains the GFFP gene. As such, the virus spread could be conveniently visualized by the appearance of the green color). Rapamycin did not show any effect on the spread of Baco-1 in permissive tumor cells (A). Original magnification: ×200.

FIG. 4. Rapamycin enhances the replication of other types of oncolytic HSVs in tumor cells that do not fully support the virus growth. In addition to Baco-1, other types of oncolytic HSVs, including FusOn-H2 (an oncolytic HSV derived from HSV-2, while Baco-1 was derived from HSV-1) and Ape-Mir3 (an oncolytic HSV that specifically targets to hepatocellular carcinoma), can also be potentiated by rapamycin when tested in resistant tumor cells. Their yield in the resistant EC9706 tumor cells was increased between 3 and 10 folds by the drug. Values represent the mean±SD of triplicate experiments. Such a result indicates that this invention applies to any types of oncolytic HSV, i.e., rapamycin and LY294002 can be used to potentiate any type of oncolytic HSV.

FIG. 5. Rapamycin potentiates the antitumor effect of oncolytic HSV against resistant tumor xenografts growing in immunodeficient animals. When tumor bearing animals were treated with rapamycin and oncolytic HSV (Baco-1) together, the therapeutic effect was significantly better than in animals treated with either of them alone [symbol] P<0.05 vs. all other groups; ★P<0.01 vs. all other groups. Two-tailed t-test.

FIG. 6. Rapamycin increases oncolytic HSV spreading within tumor xenografts. Co-administration of rapamycin with oncolytic HSV (Baco-1) to tumor-bearing animals can increase the spread of the virus within tumor tissues, as judged by visualization of tumor sections after immunohistochemical staining for GFP (A) and by counting GFP-positive tumor cells isolated from the treated tumor tissues (B). [symbol] P<0.01 vs. Baco-1 treatment alone.

### DETAILED DESCRIPTION OF THE INVENTION

The present invention relates to the design, construction, characterization and use of a novel method to significantly increase the yield and dissemination of oncolytic Herpes simplex viruses (HSVs) in resistant tumor cells. The present invention also relates to the combined administration of PI3K/AKT/mTOR pathway inhibitors (e.g., rapamycin and LY294002) and a HSV-derived oncolytic virus to either block or reverse the growth of tumors that are otherwise resistant to the therapeutic effect of either agent alone. This invention has important applications in potentiating the activity of oncolytic HSVs against difficult-to-treat human tumors and/or in preventing the emergence of resistant tumor cells during virotherapy.

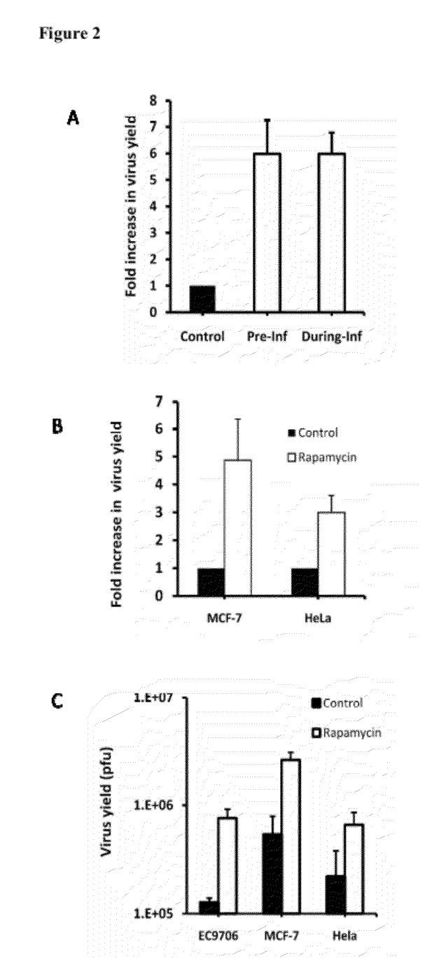

In a preferred embodiment of the present invention the inhibition of PI3K/AKT/mTOR signaling pathway improves the replication potential of oncolytic HSVs in cells that are known to be highly resistant to oncolytic HSV replication (Fu, et al., Virotherapy induces massive infiltration of neutrophils in a subset of tumors defined by a strong endogenous interferon response activity. Cancer Gene Ther. 2011 August 26. doi: 10.1038/cgt.2011.46). The presence of rapamycin or LY294002 in the medium increases the replication of Baco-1 (an oncolytic HSV) in EC9706 cells (known to be highly resistant to oncolytic HSV replication) more than 6-fold as illustrated in FIG. 2A. Pre-incubation of EC9706 cells with these drugs leads to the same level of virus increase as seen when the drugs are present during virus infection. FIG. 2B shows similar results when two other kinds of resistant tumor cell lines, namely the human breast cancer line MCF-7 and HeLa adenocarcinoma cells, are used. FIG. 2C shows the actual virus titers obtained from the resistant tumor cells before and after the drug treatment. These results illustrate that the drug-induced inhibition of PI3K/AKT/mTOR signaling pathway enhances the replication of an oncolytic HSV in tumor cells highly resistant to virus replication, and the enhancement effect could be achieved when the drugs are given before or during virotherapy.

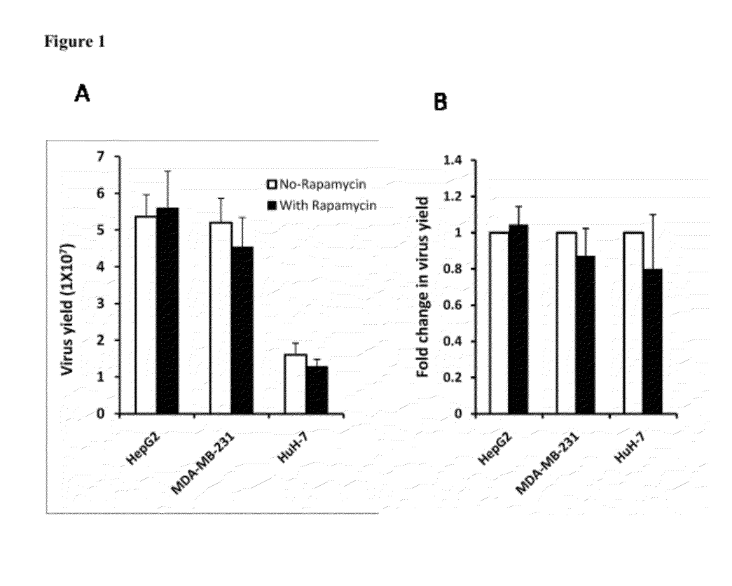

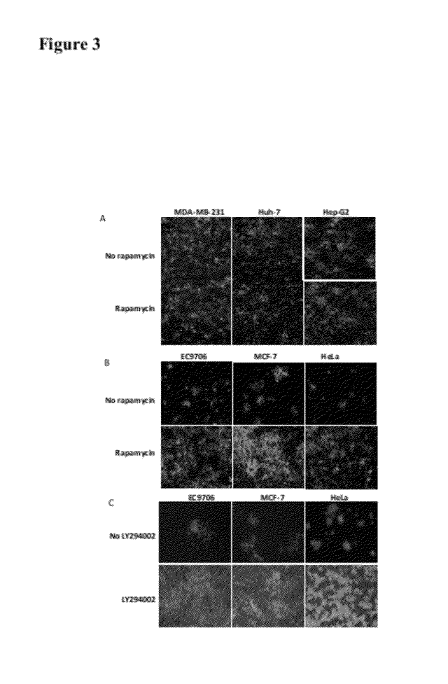

In another preferred embodiment of the present invention the use of rapamycin or LY294002 promotes the spread of oncolytic HSV in highly resistant, but not fully permissive tumor cells. Because Baco-1 contains the Green Fluorescent Protein (GFP) gene, it is possible to visualize GFP expression during virus infection and thus monitor the spread of virus among tumor cells. FIG. 3A shows that Baco-1 infects a majority of the permissive cells by 48 h, a result that is not substantially affected by the addition of rapamycin. By contrast, the virus does not spread extensively among these resistant tumor cells even at 96 h post infection. The infection foci are sparsely distributed across the cell monolayer, and many of the initially infected cells remain either as single GFP-positive cells or spread to only a few surrounding cells, as shown in FIG. 3B. In the presence of rapamycin, however, the virus spreads more widely, infecting almost the entire cell monolayer by 96 h postinfection. The effect of rapamycin on Baco-1 replication is subsequently examined in several additional cell lines and the results, together with those shown in FIGS. 1 and 2, are summarized in Table 1 below. These results demonstrate that, despite lacking any substantial effect on virus replication in fully permissive tumor cells, rapamycin enhances the replication and dissemination of an oncolytic HSV in highly resistant tumor cells.

TABLE 1
Summary of changes in virus yield in different cells
after incubation with rapamycin

| | | Relative | |
|---|---|---|---|
| Cancer cell | Tissue | permissiveness | Fold of change |
| lines | origin | to Baco-1¹ | in virus yield² |
| EC9706 | esophagus | + | 5.98 |
| MCF-7 | breast | + | 4.86 |
| HeLa | cervix | + | 3.0 |
| 293T | kidney | + | 8.01 |
| SW480 | colon | ++ | 5.31 |
| HepG2 | liver | ++++ | 1.04 |
| HuH-7 | liver | +++ | 0.8 |
| Hep3B | liver | +++ | 1.10 |
| MDA-MB-231 | breast | +++ | 0.87 |
| MDA-MB-435 | breast | ++++ | 0.6 |
| A549 | lung | +++ | 0.42 |
| DLD-1 | colon | +++ | 1.02 |
| HCT116 | colon | +++ | 0.5 |
| PANC-1 | pancreas | ++ | 0.99 |
| MPanc-96 | pancreas | +++ | 0.55 |

¹Relative permissiveness was estimated by checking for GFP positive cells 24 h after Baco-1 infection at 0.1 pfu/cell.
²Fold of change in virus yield was calculated by dividing the virus yield from the well with rapamycin with that from the well without the drug.

In another preferred embodiment of the present invention the combined administration of one or more than one drug, such as rapamycin and LY294002, a P13 kinase inhibitor, when combined with HSV-based virotherapy, enhances the therapeutic effect—while use of either agent alone produces only transient inhibitory effect. FIG. 3C shows that the upstream component of the PI3K/Akt/mTOR pathway is also involved in regulating oncolytic HSV replication in the highly resistant cancer cells. The effect of PI3K inhibitor, LY294002, on the replication of Baco-1 is examined in these cells. The three highly resistant tumor cell lines are infected with Baco-1, with or without the presence of LY294002 in the medium. As shown in FIG. 3C, LY294002 significantly enhances the replication and spread of the virus in all three cell lines. Interestingly, incubation of the infected cells with another PI3K inhibitor (wortmannin) or two Akt inhibitors (Akt Inhibitor IV and V) does not result in any significant increase in Baco-1 replication, indicating that more than one component of the PI3K/Akt/mTOR axis is involved in regulating oncolytic HSV replication in these highly resistant tumor cells, and that drugs as such rapamycin and LY294002 may be combined together with HSV-based virotherapy to enhance the therapeutic effect.

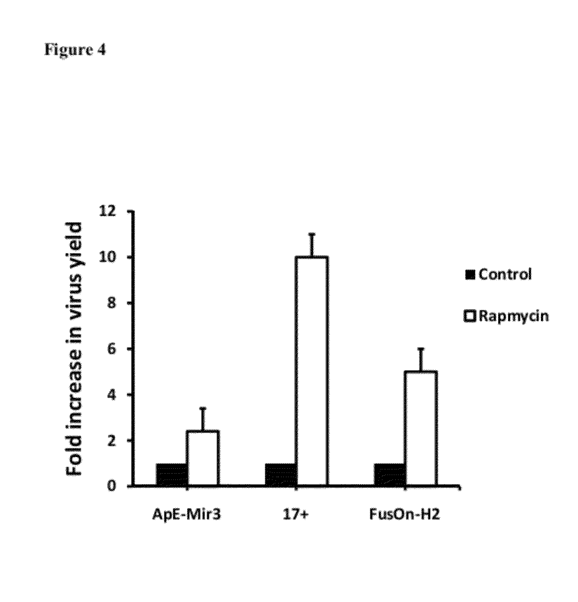

In another preferred embodiment of the present invention the potentiating effect of rapamycin on virus replication in highly resistant tumor cells is applied to other oncolytic HSVs, including strain 17 (17⁺), a wild-type HSV-1, FusOn-H2, which is constructed from an HSV-2 by mutating the N-terminal region of the ICP 10 gene (Fu, et al., A Mutant Type 2 Herpes Simplex Virus Deleted for the Protein Kinase Domain of the ICP10 Gene Is a Potent Oncolytic Virus, Mol. Ther. 2006; 13:882-90), and ApE-Mir-3, an HSV-1-based oncolytic virus in which the glycoprotein H (gH) gene is controlled by tissue-specific microRNAs (miRNAs), including let-7 and mir-122. To that end, EC9706 cells are infected with these viruses at 0.1 pfu/cell for 1 h and then cultured the cells in media with or without rapamycin at a concentration of 100 nM. The results in FIG. 4 show that rapamycin increases the replication of all three viruses. The wild type-strain, 17⁺, shows the greatest increase in virus yield (more than 10-fold) when the drug is present. Replication of the HSV-2-based oncolytic virus, FusOn-H2, is increased by approximately 5-fold, similar to those seen with Baco-1. The ApE-Mir-3 virus shows only a 2.5-fold increase, probably because of the intrinsic limitations imposed by the miRNA profiles in the tumor cells. These results suggest that the enhancement of HSV replication by rapamycin is a general phenomenon in the resistant tumor cells infected by these viruses.

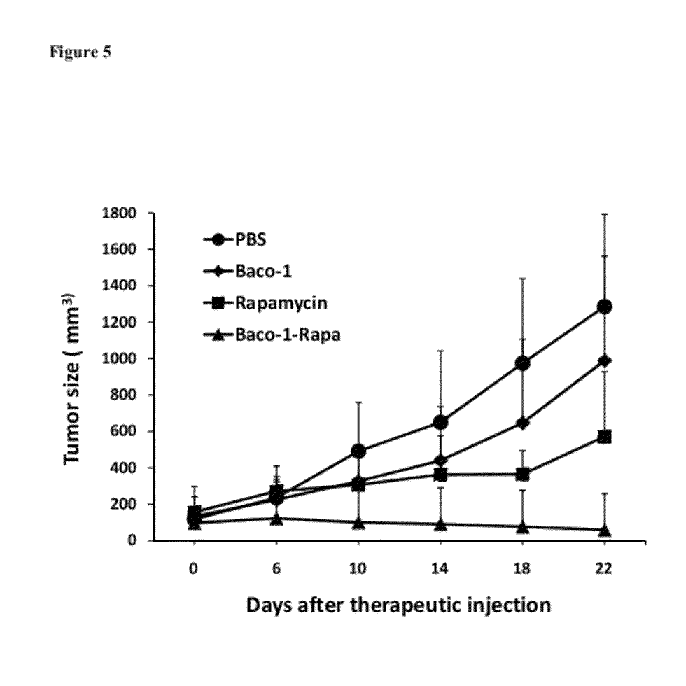

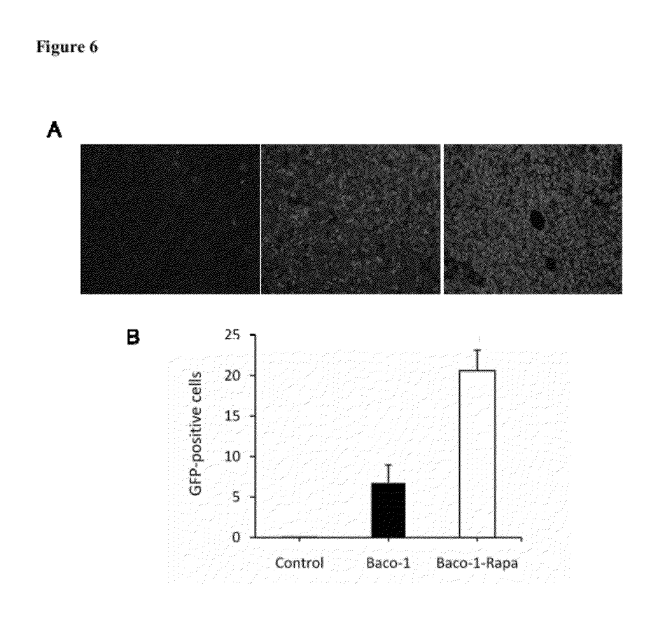

In yet another preferred embodiment of the present invention the coadministration of rapamycin significantly increases the antitumor effect of Baco-1 in vivo. To test the beneficial effect of rapamycin on the oncolytic activity of Baco-1 in vivo, tumors from the highly resistant EC9706 line of human esophageal carcinoma cells are established by implanting tumor cells into the right flank of immune-deficient mice. When tumors reach the approximate size of 5 mm in diameter, the mice are divided randomly into four groups and treated by: (i) intratumoral injection of PBS only; (ii) intratumoral injection of Baco-1; (iii) intraperitoneal administration of rapamycin; and (iv) intratumoral injection of Baco-1 and intraperitoneal administration of rapamycin. Rapamycin is given daily at the dose of 50 μg/kg body weight, a dose that had been shown to only marginally affect EC9706 tumor growth when the drug is given alone (Hou, et al., mTOR inhibitor rapamycin alone or combined with cisplatin inhibits growth of esophageal squamous cell carcinoma in nude mice, Cancer Lett. 2010; 290:248-54). Antitumor effects are assessed over 3 weeks. While Baco-1 and rapamycin given alone slowed the growth of EC9706 tumors in mice, the combined administration of these agents (Baco-1-Rapa) blocked the growth entirely as illustrated in FIG. 5. Notably, by 10-18 days postinjection, the tumors treated with Baco-1 or rapamycin alone regain their initial growth rates, whereas those exposed to Baco-1-Rapa continue to shrink over the 22-day observation period. To confirm the results presented in FIG. 5, tumors from mice 24 h after treatment are collected to determine the spread of virus within the tumor tissues. Interference from the autofluorescence emitted from tissue samples is avoided by staining the tumor sections immunohistochemically for GFP expression. As compared with tumors in the Baco-1 only group, those treated by coadministration of rapamycin and Baco-1 had visually stronger staining of GFP as evidenced in FIG. 6A. Quantification of GFP positive cells showed three times more GFP positive cells in tumors in the Baco-1-Rapa group compared with the Baco-1 only group (FIG. 6B). Thus, as in cell cultures, the coadministration of rapamycin with an oncolytic HSV in vivo potentiates the antitumor effect of virotherapy against tumor xenografts established from highly resistant human tumor cells.

### EXAMPLES

### Example 1

Rapamycin enhances oncolytic HSV replication in tumor cells that do not fully support the virus growth. A. EC9706 cells were either preincubated with rapamycin overnight (Pre-Inf) or incubated with the drug during the virus infection (During-Inf). They were then infected with Baco-1 at 0.1 pfu/cell and for 72 h. Fold increase in virus yield was calculated by dividing the yield in the control well with that in the rapamycin treated well. Rapamycin was found to increase the yield of an oncolytic HSV by almost 6-fold. B. MCF-7 and HeLa cells were infected with Baco-1 at 0.01 pfu/cell for 1 h. Then the cells were cultured in medium without (control) or with rapamycin at a concentration of 100 nM for 72 h before harvesting for virus titration. Rapamycin was found to increase the yield of an oncolytic HSV by 3-5 fold. C. without the drug treatment, the yield of oncolytic virus in these resistant tumor cells was quite low (around 1×10⁵ plaque forming units (pfu). In the presence of rapamycin, the virus yield was increased by more than a log, to almost 5×10⁶ pfu.

### Example 2

Rapamycin promotes the spread of oncolytic HSV in semipermissive tumor cells and tumor cells that do not fully support the virus growth. Three permissive tumor cells (MDA-MB-231, Huh-7 and Hep-G2 cells) are infected with Baco-1 at 0.1 pfu/cell and incubated without or with rapamycin (100 nM). Micrographs taken at 48 h postinfection did not show any effect on the spread of Baco-1 in these permissive tumor cells. In the same experiment, three highly resistant tumor cells (EC9706, MCF-7 and Hela cells) are infected with Baco-1 at 0.01 pfu/cell and incubated without or with rapamycin at a concentration of 100 nM, or LY294002 at 50 μM concentration. Both rapamycin and LY294002 were found to dramatically enhance the spread of the oncolytic HSV (Baco-1) in all the three resistant tumor cells.

### Example 3

Rapamycin enhances the replication of several other types of oncolytic HSVs in tumor cells that do not fully support the virus growth. EC9706 cells were infected with three other types of oncolytic HSVs, including FusOn-H2 (an oncolytic HSV derived from HSV-2, while Baco-1 was derived from HSV-1) and Ape-Mir3 (an oncolytic HSV specifically targets to hepatocellular carcinoma), at 0.1 pfu/cell and then incubated with medium without or with rapamycin (100 nM) for 72 h before harvesting for virus titration. Rapamycin was found to increase the yield of these viruses by 3-10 folds in this highly resistant tumor cells.

### Example 4

Rapamycin potentiates the antitumor effect of oncolytic HSV against resistant tumor xenografts growing in immunodeficient animals. Mice bearing implanted EC9706 tumors on the right flank were mocked treated (PBS), treated with either Baco-1 (1×10⁶ intratumorally) or rapamycin (50 μg/kg intraperitoneally), or treated with the combination of these agents (n=8 mice per group). Tumor size was periodically monitored after treatment. The results show that the combinatorial treatment resulted in a therapeutic effect that was significantly better than either treatment alone, indicating that rapamycin can greatly potentiate the antitumor effect of the HSV-virotherapy.

### Example 5

Rapamycin increases oncolytic HSV spreading within tumor xenografts. Mice bearing implanted EC9706 tumors were mocked treated (PBS), treated with either Baco-1 (1×10⁶ intratumorally) or rapamycin (50 μg/kg intraperitoneally), or treated with the combination of these agents. Tumor samples were collected 3 days after treatment. Because of possible interference of nonspecific autofluorescence from the tumor tissues, tumor sections were immunohistochemically stained for GFP, as a means to assess the distribution of Baco-1 within tumor masses. The first antibody is rabbit anti-GFP polyclonal antibody and the second antibody is Texas red-conjugated goat anti-rabbit IgG antibody. Co-administration of rapamycin with oncolytic HSV (Baco-1) to tumor-bearing animals can greatly increase the spread of the virus within tumor tissues, as judged by visualization of tumor sections after immunohistochemical staining for GFP and by counting GFP-positive tumor cells isolated from the treated tumor tissues.

While the invention described herein specifically focuses on use of a novel method to significantly increase the yield and dissemination of oncolytic HSVs in semipermissive tumor cells, one of ordinary skills in the art, with the benefit of this disclosure, would recognize the extension of the approach to other combinatorial treatment regimens for the treatment of resistant tumors in a clinical setting.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no embodiments disclosed herein are intended to limit the scope of the claims of this invention. It is therefore evident that the particular illustrative embodiments disclosed above may be altered or modified and all such variations are considered within the scope and spirit of the present invention.

What is claimed is:

1. A method of treating tumor cells in an HSV-based oncolytic virotherapy, comprising:
administering a therapeutic composition comprising one or more PI3K/AKT/mTOR pathway inhibitors; and
administering a therapeutic composition comprising an HSV derived oncolytic virus.

2. The method of claim 1, wherein the therapeutic composition comprising one or more PI3K/AKT/mTOR pathway inhibitors is administered prior to administering the therapeutic composition comprising an HSV derived oncolytic virus.

3. The method of claim 1, wherein the therapeutic composition comprising one or more PI3K/AKT/mTOR pathway inhibitors is administered with the therapeutic composition comprising an HSV derived oncolytic virus.

4. The method of claim 1, wherein the therapeutic composition comprising one or more PI3K/AKT/mTOR pathway inhibitors and therapeutic composition comprising an HSV derived oncolytic virus are administered to resistant tumor cells.

5. The method of claim 4, wherein the therapeutic composition comprising one or more PI3K/AKT/mTOR pathway inhibitors increases the permissiveness of the resistant tumor cells to the HSV derived oncolytic virus.

6. The method of claim 1, wherein the therapeutic composition comprising PI3K/AKT/mTOR pathway inhibitors comprises one or any combination of rapamycin, LY294002, wortmannin, AKT Inhibitor IV, or AKT Inhibitor V.

7. The method of claim 1, wherein the HSV derived oncolytic virus is Baco-1, FusOn-H2, or ApE-Mir-3.

8. The method of claim 1, wherein the HSV derived oncolytic virus blocks or reverses growth of resistant tumors.

9. A composition for treating cancer in an HSV-based oncolytic therapy, the composition comprising
an HSV derived oncolytic virus; and
one or more PI3K/AKT/mTOR pathway inhibitors.

10. The composition of claim 9, wherein the composition increases permissiveness of resistant tumor cells to the HSV derived oncolytic viruses.

11. The composition of claim 9, wherein the one or more PI3K/AKT/mTOR pathway inhibitors comprises one or any combination of rapamycin, LY294002, wortmannin, AKT Inhibitor IV, or AKT Inhibitor V.

12. The composition of claim 9, wherein the HSV derived oncolytic virus is Baco-1, FusOn-H2, or ApE-Mir-3.

13. The composition of claim 9, wherein the composition blocks or reverses growth of tumors from resistant tumor cells.

14. A composition adapted for use in conjunction with one or more PI3K/AKT/mTOR pathway inhibitors, comprising:
an HSV derived oncolytic virus.
